(19) United States
(12) Patent Application Publication
(10) Pub. No.: US 2026/0224113 A1
(43) Pub. Date: Aug. 6, 2026

(54) DEVICES AND METHODS FOR OBTAINING EMERGENT FACTORS OF USERS

(71) Applicant: EMERJA CORPORATION, Monte Sereno, CA (US)

(72) Inventors: Guy Miller, Monte Sereno, CA (US); Daniel Hansen, Santa Cruz, CA (US); Wei-Meng Chee, Belmont, CA (US)

(21) Appl. No.: 19/146,445

(22) PCT Filed: Jan. 10, 2024
(86) PCT No.: PCT/US2024/011088

§ 371 Date: Jul. 8, 2025

Related U.S. Application Data
(60) Provisional application No. 63/479,671, filed on Jan. 12, 2023.
(60) Provisional application No. 63/601,909, filed on Nov. 22, 2023.
(60) Provisional application No. 63/612,832, filed on Dec. 20, 2023.

(51) Int. Cl. A61B 5/01; A61B 5/00

(52) U.S. Cl. CPC A61B 5/01; A61B 5/0008; A61B 5/4857; A61B 5/4866; A61B 5/681; A61B 2560/0252

(57) ABSTRACT

Disclosed are devices and methods for measuring a plurality of data indicative of at least one emergent factor of a user. In one aspect, an exemplary device is wearable by the user and includes a circuit board located within a housing, the circuit board having a first side of the circuit board opposite a second side of the circuit board; a first sensor located on the first side of the circuit board; and a second sensor located on the second side of the circuit board, wherein the first sensor and the second sensor are thermally decoupled, and wherein the first and second sensors measure said at least one emergent factor of the user. In one embodiment, an exemplary device is configured to perform simultaneous, continuous measurement of skin surface temperature and ambient temperature.

### CROSS-REFERENCE TO RELATED APPLICATIONS

This application claims priority to U.S. Provisional Application No. 63/479671 filed Jan. 12, 2023, U.S. Provisional Application No. 63/601909 filed Nov. 22, 2023, and U.S. Provisional Application No. 63/612832 filed Dec. 20, 2023, the content of each of which is incorporated by reference in its entirety.

### BACKGROUND

### Field of the Disclosed Technology

The disclosed technology generally relates to systems for, wearable devices for, and methods of obtaining emergent properties of a complex adaptive system, such as a biological system, an organism, or for example a human, or a non-biological system. More specifically, the disclosed technology relates to physiological assessment of a human user by obtaining emergent factors of the user with a noninvasive wearable device for maintaining or improving health. The disclosed technology also generally relates to an automatically adjusting or manually adjustable band.

### Description of the Related Art

Known health monitoring wearable devices may utilize digital temperature sensors to perform high-accuracy measurements of the skin temperature of a user and the temperature of the surrounding environment. However, such known devices for monitoring users are inadequate. For example, there is a need for improved thermal design to maximize the thermal impedance, or minimize thermal crosstalk, between different temperature sensors in a health monitoring wearable device. Moreover, there is a need for health monitoring wearable devices that are capable of obtaining emergent factors of a user which are indicative of the user's homeostasis and health state. Further details regarding emergent factors indicative of a user's homeostasis and health state can be found in International Patent Application No. PCT/US2021/048053, titled “SYSTEMS AND METHODS FOR MEASURING, LEARNING, AND USING EMERGENT PROPERTIES OF COMPLEX ADAPTIVE SYSTEMS” and filed on Aug. 27, 2021, the disclosures of which are incorporated herein by reference.

A historic perspective sheds light on the manifold advantages of the disclosed technology. Known devices for and methods of monitoring biological systems, and known systems for maintaining or improving health are inadequate. For example, known wearable devices generally exist in the off-the-shelf market; however, many patients and users are excluded from that market. Furthermore, and as a further example, known wearable devices are generally “one size fits all”; however, many patients and users cannot be accommodated by such devices. Additionally, wearable devices generally will accomplish their intended functions only when worn as intended and serve little or no function if not worn substantially continually. There are usually critical moments in which the wearable device should be worn, but off-the-shelf market and one size fits all devices may be unworn at those moments and have features that often motivate a patients and users to remove the wearable device.

### SUMMARY

Designs of, method for making, methods of adjusting, and methods of improving compliant use and extended use of an adjustable wearable device are disclosed. The disclosed technology provides a device that is wearable by a user, the device comprising a band configured to be worn in substantially complete cross-sectional contact with a biological compartment of the user; a tension mechanism connected to the band, the tension mechanism configured to allow a circumference of the band (i) to expand to accommodate ingress of fluid into the biological compartment and (ii) to contract to accommodate egress of fluid out of the biological compartment, such that the band maintains substantially complete cross-sectional contact with the biological compartment of the user; and a sensor connected to the band, the sensor configured to collect a plurality of patient data. Method of collecting and distributing data, collected via the device, are also disclosed. In certain embodiments of the devices and of the methods disclosed, the data relates to the health capacity of the user. In certain embodiments of the devices and of the methods disclosed, the data relates to the heat flux of the user. In certain embodiments of the devices and of the methods disclosed, the data relates to the heat flux of the user over multiple circadian cycles.

The disclosure provides designs and methods of manufacturing a device for continuously and contextually characterizing an individual's metabolic state by measuring their thermal signature to assess what is referred to as a thermoregulatory phenotype. Changes relative to this phenotype are sensitive indicators of change in health state. The device is designed and configured such that it delivers, in human use, general associations between an individual's thermal signature and physiologic reserve. Additional information is available within the thermal signature to an actionable assessment of readiness. Clinical studies are designed to gather data that will serve as a novel vital sign of homeostasis as well as an aggregate health signature, with applicability to early detection of many disease states, management of individual wellness and readiness.

In one aspect, the disclosed technology relates to a device configured to measure a plurality of data indicative of at least one emergent factor of a user, the device comprising: a circuit board located within a housing, the circuit board having a first side of the circuit board opposite a second side of the circuit board; a first sensor located on the first side of the circuit board; and a second sensor located on the second side of the circuit board, wherein the first sensor and the second sensor are thermally decoupled, and wherein the first and second sensors measure said at least one emergent factor of a user.

In some embodiments, the first sensor is a heat sensor and the second sensor is a heat sensor. In some embodiments, the first sensor measures skin temperature. In some embodiments, the second sensor measures ambient temperature. In some embodiments, the device further includes a battery. In some embodiments, the battery is positioned between the first sensor and the second sensor. In some embodiments, the battery serves to thermally isolate the first sensor from the second sensor. In some embodiments, the second sensor is connected to a thermal ring. In some embodiments, the thermal ring contacts the user. In some embodiments, the device further includes a processor and a firmware.

For example, an exemplary device is configured to measure a plurality of data indicative of at least one emergent factor of a user, the device comprising: a circuit board; a first channel of the circuit board configured to measure ambient temperature; and a second channel of the circuit board configured to measure skin temperature of the user; wherein the first channel and the second channel are decoupled. In some embodiments, the device further includes a battery, a processor, and a firmware.

In some embodiments, the device is wearable by a user. In some embodiments, the device further includes a band configured to be worn in substantially complete cross-sectional contact with a biological compartment of the user. The band may be automatically adjusted or manually adjusted, such as described in U.S. Provisional Application No. 63/371365, titled “A NONINVASIVE WEARABLE FOR THE PHYSIOLOGICAL ASSESSMENT OF PHYSICAL AND COGNITIVE READINESS FOR MILITARY TASKS” and filed on Aug. 12, 2022, the disclosures of which are incorporated herein by reference. For example, a tension mechanism may be connected to the band, the tension mechanism configured to allow a circumference of the band to expand to accommodate ingress of fluid into the biological compartment and to contract to accommodate egress of fluid out of the biological compartment, such that the band maintains substantially complete cross-sectional contact with the biological compartment of the user. Sensors configured to collect user data may be connected to the band.

In another aspect, the disclosed technology relates to a method of measuring a plurality of data indicative of at least one emergent factor of a user, the method comprising: measuring ambient temperature of the user through a first sensor; and measuring skin temperature of the user through a second sensor, wherein the first sensor and the second sensor are decoupled.

In some embodiments, the method further includes: estimating heat elimination of a biological system over time based on differential ambient temperature; estimating heat production of the biological system over time based on skin temperature; and estimating basal metabolic status of the biological system based on temporal alignment of heat elimination and heat production.

In some embodiments, the method further includes: obtaining a quasiperiodic rhythm of a biological system based on ambient temperature and skin temperature, wherein the quasiperiodic rhythm is of seconds-timescale, of minutes-timescale, ultradian, circadian, circalunar, or of yearly timescale.

In some embodiments, the method further includes: obtaining a variability of the quasiperiodic rhythm across a predetermined amount of time; and determining a health capacity based on the variability of the quasiperiodic rhythm.

In some embodiments, the method further includes: estimating heat elimination of a biological system over time based on differential ambient temperature; estimating heat production of the biological system over time based on skin temperature; estimating a basal metabolic status of the biological system based on temporal alignment of heat elimination and heat production; and determining a health capacity by applying a time-dependent function to the estimated basal metabolic status, wherein the time-dependent function is derived from the quasiperiodic rhythm of the biological system.

In some embodiments, the data relates to the health capacity of the user. In some embodiments, the plurality of data comprises heat flux data. In some embodiments, the data relates to the heat flux of the user. In some embodiments, the data relates to the heat flux of the user over multiple circadian cycles. In some embodiments, at least one health capacity is a basal metabolic status, and at least one emergent factor is a temporal alignment of heat production and heat elimination. In some embodiments, the temporal alignment is related to at least one quasiperiodic rhythm of a biological system. In some embodiments, the at least one quasiperiodic rhythm is a circadian rhythm.

In some embodiments, the device continuously and contextually characterizes a user's metabolic state by measuring the user's thermal signature and assessing the user's thermoregulatory status. Changes relative to the user's thermoregulatory status are sensitive indicators of change in the user's health state. The device may be configured such that it determines general associations between a user's thermal signature and physiologic reserve. The device may indicate a vital sign of homeostasis or a health signature of the user, and may be utilized in early detection of disease states and management of individual wellness.

In another aspect, the disclosed technology relates to a device configured to measure a plurality of data indicative of at least one emergent factor of a user, the device comprising: a housing; a circuit board located within the housing, the circuit board having a first side of the circuit board opposite a second side of the circuit board; a first sensor located on the first side of the circuit board and coupled to a first thermal window; and a second sensor located on the second side of the circuit board and coupled to a second thermal window; wherein the first sensor and the second sensor are decoupled, wherein the first thermal window and the second thermal window are decoupled, and wherein the second sensor is coupled to the circuit board by a flexible connector.

In some embodiments, the first sensor is a heat sensor and the second sensor is a heat sensor.

In some embodiments, the first sensor measures skin temperature.

In some embodiments, the second sensor measures ambient temperature.

In some embodiments, the first thermal window contacts the user and the second thermal window contacts ambient air.

In some embodiments, the first thermal window and the second thermal window are thermally conductive and permit a long-range Bluetooth signal to be transmitted through the device.

In some embodiments, the first thermal window and the second thermal window have a thickness configured to permit an efficient first sensor response and an efficient second sensor response.

In some embodiments, the first thermal window and the second thermal window are made of a dielectric material.

In some embodiments, the dielectric material is sapphire.

In some embodiments, the dielectric material is diamond.

In some embodiments, the dielectric material is synthetic sapphire or synthetic diamond.

In some embodiments, the device further includes a battery.

In some embodiments, the first sensor and the second sensor are thermally decoupled.

In some embodiments, the first sensor and the second sensor are configured to measure data indicative of the at least one emergent factor of the user.

In some embodiments, the battery is positioned between the first sensor and the second sensor.

In some embodiments, the battery serves to thermally isolate the first sensor from the second sensor.

In some embodiments, the device further includes a processor and a firmware.

In another aspect, the disclosed technology relates to a device configured to measure a plurality of data indicative of at least one emergent factor of a user, the device comprising: a circuit board; a first channel of the circuit board configured to measure ambient temperature; and a second channel of the circuit board configured to measure skin temperature of the user; wherein the first channel and the second channel are decoupled, and wherein the first channel is coupled to a first thermal window that contacts the user and the second channel is coupled to a second thermal window that contacts ambient air.

In some embodiments, the device further includes a battery, a processor, and a firmware.

In some embodiments, the first thermal window and the second thermal window are decoupled.

In some embodiments, the first thermal window and the second thermal window are made of a dielectric material.

In another aspect, the disclosed technology relates to a method of measuring a plurality of data indicative of at least one emergent factor of a user, the method comprising: measuring ambient temperature of the user through a first sensor; and measuring skin temperature of the user through a second sensor, wherein the first sensor and the second sensor are decoupled, and wherein the first sensor is coupled to a first thermal window and the second sensor is coupled to a second thermal window.

In some embodiments, the method further comprises: estimating heat elimination of a biological system over time based on differential ambient temperature; estimating heat production of the biological system over time based on skin temperature; and estimating basal metabolic status of the biological system based on temporal alignment of heat elimination and heat production.

In some embodiments, the method further comprises: obtaining a quasiperiodic rhythm of a biological system based on ambient temperature and skin temperature, wherein the quasiperiodic rhythm is of seconds-timescale, of minutes-timescale, ultradian, circadian, circalunar, or of yearly timescale.

In some embodiments, the method further comprises: obtaining a variability of the quasiperiodic rhythm across a predetermined amount of time; and determining a health capacity based on the variability of the quasiperiodic rhythm.

In some embodiments, the method further comprises: estimating heat elimination of a biological system over time based on differential ambient temperature; estimating heat production of the biological system over time based on skin temperature; estimating a basal metabolic status of the biological system based on temporal alignment of heat elimination and heat production; and determining a health capacity by applying a time-dependent function to the estimated basal metabolic status, wherein the time-dependent function is derived from the quasiperiodic rhythm of the biological system.

In some embodiments, the plurality of data comprises heat flux data.

In some embodiments, at least one health capacity is a basal metabolic status, and at least one emergent factor is a temporal alignment of heat production and heat elimination.

In some embodiments, the temporal alignment is related to at least one quasiperiodic rhythm of a biological system.

In some embodiments, the at least one quasiperiodic rhythm is a circadian rhythm.

In another aspect, the disclosed technology relates to a device configured to measure a plurality of data indicative of at least one emergent factor of a user, the device comprising: a housing; a circuit board located within the housing, the circuit board having a first side of the circuit board opposite a second side of the circuit board; a first sensor located on the first side of the circuit board; a second sensor located on the second side of the circuit board; and a battery positioned between the first sensor and the second sensor, wherein a first isolation material thermally decouples the first sensor and/or the second sensor from the circuit board and the battery.

In some embodiments, the first sensor is coupled to a first thermal window.

In some embodiments, the second sensor is coupled to a second thermal window.

In some embodiments, the first thermal window and the second thermal window are decoupled.

In some embodiments, the first thermal window contacts the user and the second thermal window contacts ambient air.

In some embodiments, the first thermal window and the second thermal window are thermally conductive and permit Bluetooth signals to be transmitted through the device.

In some embodiments, the first thermal window and the second thermal window have a thickness configured to permit an efficient first sensor response and an efficient second sensor response.

In some embodiments, the first thermal window and the second thermal window are made of a dielectric material.

In some embodiments, the dielectric material is sapphire.

In some embodiments, the dielectric material is diamond.

In some embodiments, the dielectric material is synthetic.

In some embodiments, the first sensor is a heat sensor and the second sensor is a heat sensor.

In some embodiments, the first sensor and the second sensor are thermally decoupled.

In some embodiments, the first sensor measures skin temperature.

In some embodiments, the second sensor measures ambient temperature.

In some embodiments, the first sensor and the second sensor are configured to measure data indicative of the at least one emergent factor of the user.

In some embodiments, the device further includes a processor and a firmware.

In some embodiments, the first isolation material is located on the first side of the circuit board and/or on the second side of the circuit board.

In some embodiments, the first isolation material comprises: a three-dimensional (3D) printed plastic insulation material, a reflective foil, a reflective thermally super-insulating material, or any combination thereof.

In some embodiments, the three-dimensional (3D) printed plastic insulation material is configured to minimize thermal transfer between the first sensor and/or the second sensor and the circuit board and the battery.

In some embodiments, the reflective foil and/or the reflective thermally super-insulating material is configured to reflect away radiative heat transfer between the first sensor and/or the second sensor and the circuit board and the battery.

In some embodiments, at least a face of the housing comprises a second isolation material, a mesh of air pockets, or any combination thereof.

In some embodiments, the second isolation material and/or the mesh of air pockets is configured to minimize thermal transfer through peripheries of the device.

In some embodiments, the second isolation material is a 3D printed plastic insulation material.

In another aspect, the disclosed technology relates to a device configured to measure a plurality of data indicative of at least one emergent factor of a user, the device comprising: a housing; a circuit board located within the housing; a first channel of the circuit board configured to measure skin temperature of the user; and a second channel of the circuit board configured to measure ambient temperature; wherein the first channel and the second channel are decoupled, and wherein at least a face of the housing comprises: an isolation material, a mesh of air pockets, or any combination thereof.

In some embodiments, the isolation material and/or the mesh of air pockets is configured to minimize thermal transfer through peripheries of the device.

In some embodiments, the isolation material is a 3D printed plastic insulation material.

In some embodiments, the device further includes a battery, a processor, a firmware, or any combination thereof.

In some embodiments, the first channel is coupled to a first thermal window that contacts the user and the second channel is coupled to a second thermal window that contacts ambient air.

In some embodiments, the first thermal window and the second thermal window are decoupled.

In some embodiments, the first thermal window and the second thermal window are thermally conductive and permit Bluetooth signals to be transmitted through the device.

In some embodiments, the first thermal window and the second thermal window have a thickness configured to permit an efficient first sensor response and an efficient second sensor response.

In some embodiments, the first thermal window and the second thermal window are made of a dielectric material.

In some embodiments, the dielectric material is sapphire.

In some embodiments, the dielectric material is diamond.

In some embodiments, the dielectric material is synthetic.

In some embodiments, a first sensor is coupled to the first thermal window.

In some embodiments, a second sensor is coupled to the second thermal window.

In some embodiments, the first sensor is a heat sensor and the second sensor is a heat sensor.

In some embodiments, the first sensor and the second sensor are thermally decoupled.

In some embodiments, the first sensor measures skin temperature.

In some embodiments, the second sensor measures ambient temperature.

In some embodiments, the first sensor and the second sensor are configured to measure data indicative of the at least one emergent factor of the user.

In another aspect, the disclosed technology relates to a method of measuring a plurality of data indicative of at least one emergent factor of a user, the method comprising: measuring ambient temperature and skin temperature of the user using the device according to any of the devices disclosed above.

In some embodiments, the method further includes: estimating heat elimination of a biological system over time based on differential ambient temperature; estimating heat production of the biological system over time based on skin temperature; and estimating basal metabolic status of the biological system based on temporal alignment of heat elimination and heat production.

In some embodiments, the method further includes: obtaining a quasiperiodic rhythm of a biological system based on ambient temperature and skin temperature, wherein the quasiperiodic rhythm is of seconds-timescale, of minutes-timescale, ultradian, circadian, circalunar, or of yearly timescale.

In some embodiments, the method further includes: obtaining a variability of the quasiperiodic rhythm across a predetermined amount of time; and determining a health capacity based on the variability of the quasiperiodic rhythm.

In some embodiments, the method further includes: estimating heat elimination of a biological system over time based on differential ambient temperature; estimating heat production of the biological system over time based on skin temperature; estimating a basal metabolic status of the biological system based on temporal alignment of heat elimination and heat production; and determining a health capacity by applying a time-dependent function to the estimated basal metabolic status, wherein the time-dependent function is derived from the quasiperiodic rhythm of the biological system.

In some embodiments, the plurality of data comprises heat flux data.

In some embodiments, at least one health capacity is a basal metabolic status, and at least one emergent factor is a temporal alignment of heat production and heat elimination.

In some embodiments, the temporal alignment is related to at least one quasiperiodic rhythm of a biological system.

In some embodiments, the at least one quasiperiodic rhythm is a circadian rhythm.

### BRIEF DESCRIPTION OF THE DRAWINGS

FIG. 1 schematically illustrates an example of how a disclosed device can be used in a system that detects, processes, communicates and displays user data and health information.

FIG. 2 shows an example device of the disclosed technology, electronics of the example device, and a cross-sectional view of the example device.

FIG. 3 illustrates cross-sectional views of an example device of the disclosed technology and signals measured by the example device.

FIG. 4 illustrates cross-sectional views of an example device of the disclosed technology.

FIG. 5 illustrates a plan view and cross-sectional views of an example device of the disclosed technology.

FIG. 6 shows an embodiment of a device of the disclosed technology.

FIG. 7 schematically illustrates certain components of an example device of the disclosed technology.

FIG. 8 shows certain components of an example device of the disclosed technology.

FIG. 9 schematically illustrates certain electronics of an example device of the disclosed technology.

FIG. 10 shows an embodiment of a device of the disclosed technology.

FIG. 11 illustrates a plan view and cross-sectional views of an example device of the disclosed technology

FIG. 12 schematically illustrates certain components of an example device of the disclosed technology.

FIG. 13 schematically illustrates certain components of example devices of the disclosed technology and how they are assembled.

FIG. 14 schematically illustrates certain components of example devices of the disclosed technology and how they are assembled.

FIG. 15 shows example devices of the disclosed technology.

FIG. 16 shows example devices of the disclosed technology.

FIG. 17 shows example devices of the disclosed technology.

FIG. 18 schematically illustrates a cross-sectional view of an example device of the disclosed technology.

FIG. 19 schematically illustrated certain components of an example device of the disclosed technology and how they are assembled.

FIG. 20 schematically illustrates certain components of example devices of the disclosed technology.

FIG. 21 schematically illustrates certain components of example devices of the disclosed technology.

FIG. 22 schematically illustrates certain components of example devices of the disclosed technology and how they are assembled.

FIG. 23 schematically illustrates certain components of example devices of the disclosed technology and how they are assembled.

FIG. 24, schematically illustrates certain components of example devices of the disclosed technology and their specifications.

FIG. 25 schematically illustrates certain components of example devices of the disclosed technology and their specifications.

FIG. 26 schematically illustrates certain components of example devices of the disclosed technology and their specifications.

FIG. 27 illustrates a plan view and cross-sectional views of an example device of the disclosed technology.

FIG. 28 illustrates a plan view and cross-sectional views of an example device of the disclosed technology.

FIG. 29 illustrates a plan view and cross-sectional views of an example device of the disclosed technology.

### DETAILED DESCRIPTION OF THE PREFERRED EMBODIMENTS

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part incorporated herein by reference in their entireties for the purposes indicated by the context of their citation herein. However, the citation of any document is not to be construed as an admission that it is prior art with respect to the present disclosure.

Currently there is not an agreed upon measure of health. Health is frequently defined as the absence of disease (symptoms). Disease metrics are lagging indicators of failing health, and thus do not reflect health in an affirmative sense, and are not in and of themselves optimizable vis-à-vis real health, not disease, outcomes. For example, a challenge of a molecular biomarker is in establishing its significance relative to known physiology and homeostasis.

The generation, analysis, and use of data relating to the health condition or health capacity of biological systems (e.g., a human user) has been explored. More specifically, the use of sensors, and combinations of sensors, for capturing data related to the health capacity of biological systems has also been explored. Health capacity can refer to the resilience (adaptivity) of a system expressed primarily by its ability to persist or achieve some core function. To assess the health capacity of a system, one may interpret the emergent factors of the system. Emergent factors may refer to events, deviations from norm or other time dependent patterns in some measurable parameter of the system that can be observed directly or indirectly. Emergent factors or properties may also refer to properties of a biological system that are not readily predictable from the functions of the component parts of the system. Examples of emergent properties can include amphotericity, conductivity, solvation capacity, ion mobility, oxidation-reduction potential, ligan association, hydration, electrolysis, thermal conductivity, heat capacity, thermal absorptivity, adhesion, cohesion, transparency, turbidity, incompressibility, polarity, dipolarity, dipole movement, diamagnetism, voltage range of the liquid phase, temperature range of the liquid phase, abundancy, and speciation, flux of energy, momentum, particles or other substances, heat elimination, either as an absolute, statis value of heat elimination or as a periodic function, for example, a circadian periodicity of heat elimination.

Physicists have encountered the problem of emergent factors before (for example, magnetism) and have concluded that it may be advantageous to identify a thermodynamic parameter which summarizes the order, rather than to attempt to measure molecular details of that order directly. In fact, all order is associated with missing energy. (See, for example, https://en.wikipedia.org/wiki/Latent_heat). For example, when studying complex materials, physicists look for anomalous specific heats as the bellwether of hidden organization. Landau defined the t order parameter (See, for example, https://en.wikipedia.org/wiki/Landau_theory): a useful mathematical device which quantifies the thermodynamic character and robustness of the underlying order. Our insight is based, in part, on the concept that the organization of living systems has associated thermodynamic signatures analogous to order parameters. And, only these biological order parameters will enable highly accurate learning with small sample sizes. Furthermore, it is likely that such a thermal signature may inform us of the robustness of biological order, physiologic reserve and health state.

The underlying health condition can be monitored or assessed by use of a wearable device that can collect and/or monitor the health capacity of biological systems. It may include at least one wearable thermodynamic sensor that can be configured to measure an emergent factor of the human, wherein the emergent factor is the temporal alignment of heat production and heat elimination of the human, the temporal alignment relating to the circadian rhythm of the human, and based on the emergent factor, generate measured data comprising heat flux data over time. The wearable device may also capture heat flux data, wherein at least one health capacity is a basal metabolic status, and at least one emergent factor is the temporal alignment of heat production and heat elimination of the biological system. The wearable device may include an array of sensors that record health metrics and capture the data. The wearable device may continuously record select “energy signatures” metrics or indicators of health for the subject. In some embodiments, the wearable device requires low cost and low power, enabling accessibility and continuous data capture in real-time. In some embodiments, the wearable device comprises a multi-modality sensor system that measures electrochemical, mechanical, structural, thermal, and/or energetic properties reflective of homeostasis and cell physiology. The wearable device can comprise any number of sensors.

In some aspects, the disclosed wearable device is designed and configured to quantify physiologic energy outputs (for example, peripheral heat and physical activity). This device is benchmarked against gold-standard physiologic endpoints in multiple human studies. Metrics for such benchmarking involve a signal with high accuracy with training sets as small as 25 samples. A robust structure is identified in human heat signatures and serves as a direct measure of the autonomic processes underlying homeostasis (i.e., biological organization). Specifically, the device provides a means for the non-invasively detection of a thermal signature of an inflammatory cascade before any change in core temperature. This observation has ramifications from the perspectives of thermal physics, and transformational biological applications.

In some aspects, the wearable device described herein utilizes a physical model of temperature homeostasis, inspired by the function of the hypothalamus, to interpret the health significance of an individual's thermal signature. By measuring the principal data streams which the hypothalamus integrates (heat and body temperature), the device allows for the characterizing of the basis of homeostasis and physiologic reserve—including differences between the sexes—and for defining gender-specific metrics that are relevant to trauma injury treatment. The wearable device continuously and contextually measures these principal data streams moderated by the hypothalamus, and provides a means for characterizing both individuals and gender groups by measuring their thermal signature to assess what we call a thermoregulatory phenotype.

In some aspects, the disclosed technology is based, in part, on the utilization of a novel physical model of temperature homeostasis, providing a means for understanding and/or interpreting the health significance of an individual's thermal signature (thermal phenotype) and for acting upon that interpretation in a variety of ways. The non-invasive wearable device continuously senses thermal signature of body heat, distinct from and superior to simple skin thermometry, and requires no charge or battery replacement for periods as long as several months. Because the disclosed technology measures body heat, which is fundamentally related to temperature homeostasis, it avoids challenges in using the typical molecular biomarker.

In some aspects, the disclosure provides devices and methods for continuously and contextually characterizing an individual's metabolic state by measuring their thermal signature to assess what is referred to as a thermoregulatory phenotype. Changes relative to this phenotype are sensitive indicators of change in health state. The device is designed and configured such that it delivers, in human use, general associations between an individual's thermal signature and physiologic reserve. Additional information is available within the thermal signature to an actionable assessment of health. Clinical studies are designed to gather data that will serve as a novel vital sign of homeostasis as well as an aggregate health signature, with applicability to early detection of many disease states, management of individual wellness.

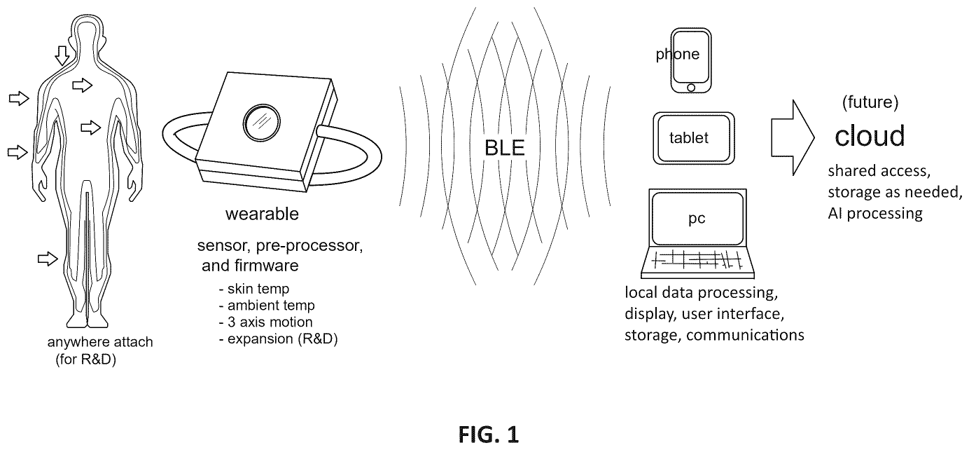

For example, as shown in FIG. 1, a wearable device may comprise a wireless transmission module to transmit data to be processed, analyzed and displayed on a smartphone, a tablet or a personal computer, which may include a memory that stores measurement data for more than a week, a month, three months or six months. Alternatively, the data may be processed, analyzed and stored on a cloud server operably connected with the smartphone, tablet or personal computer. The device may comprise a battery which can last for about six months when operating at a condition that transmits wireless signals at 1-second, 5-second, 10-second, 30-second or 1-minute intervals. The device may comprise a charge indicator for battery life. The device may transmit information regarding battery life to be displayed on the smartphone, tablet or personal computer. The device may be controlled through the smartphone, tablet or personal computer via a user interface. For example, the signal transmission intervals may be adjusted. The device may comprise a LED which indicates the status of communication or operation, or alerts and error. The device may be water resistant. The device may be wearable by an organism, such as by a person, and may be mounted to the arm, chest, leg, abdomen, or anywhere on the body.

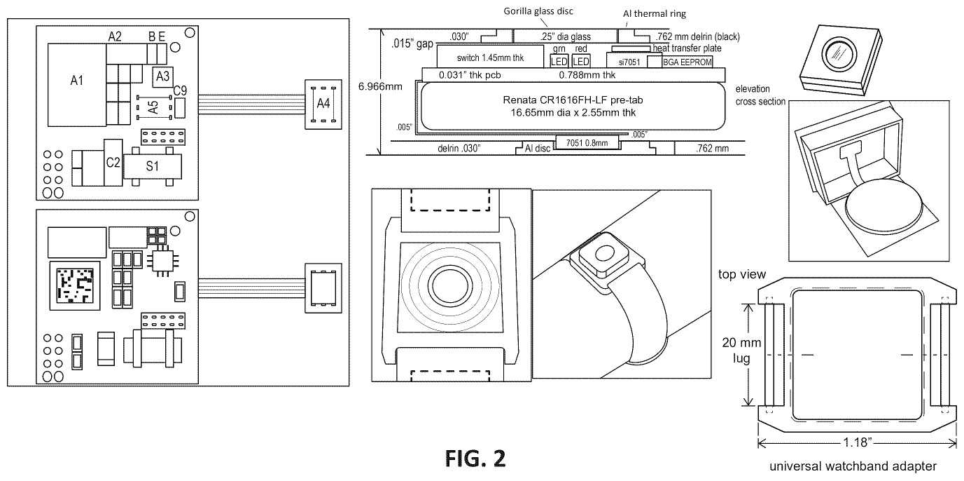

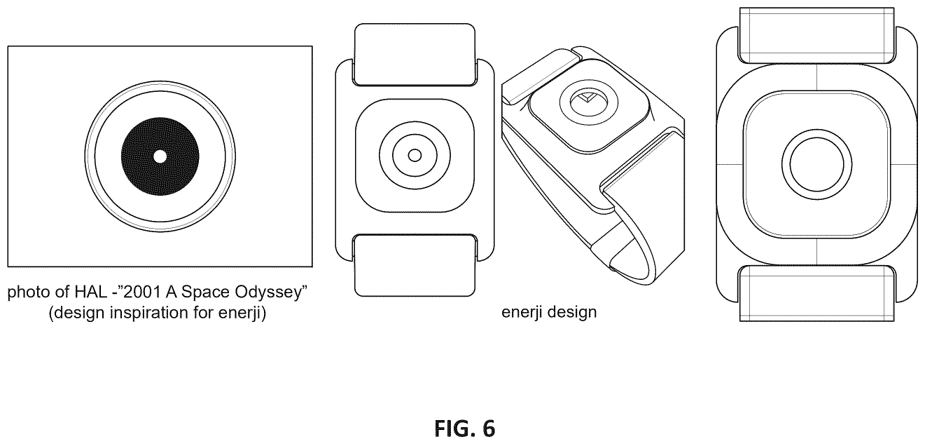

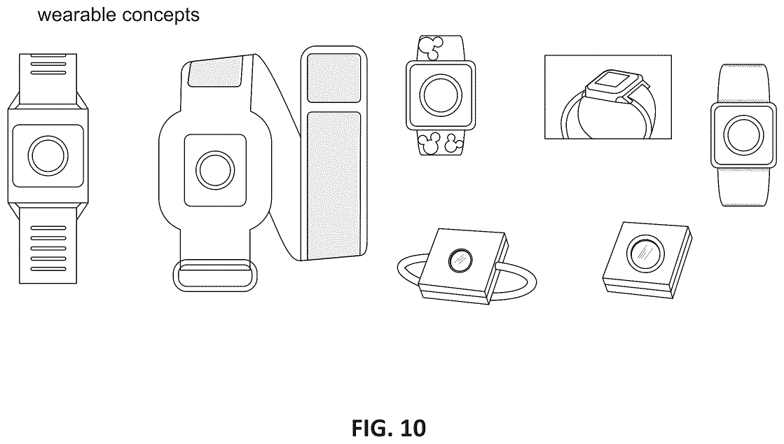

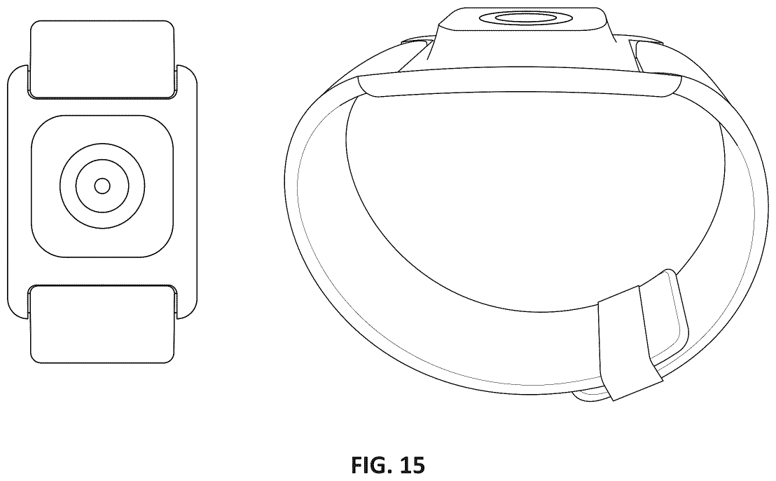

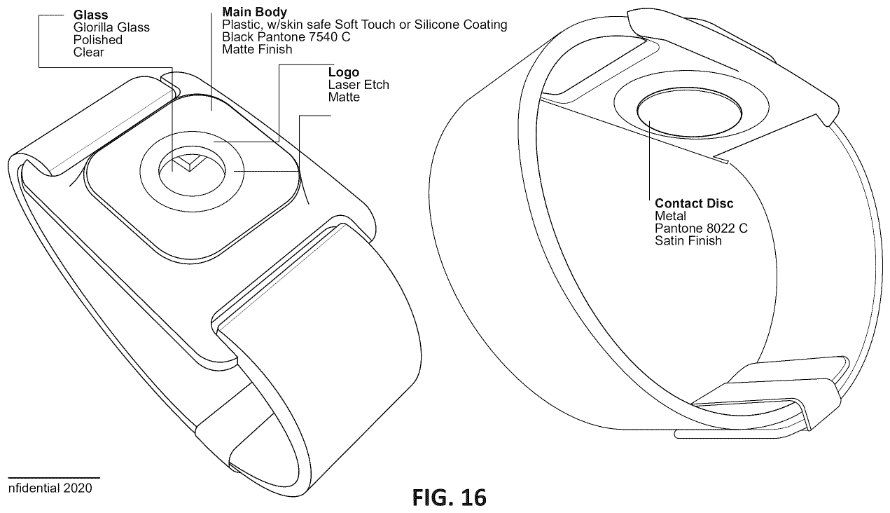

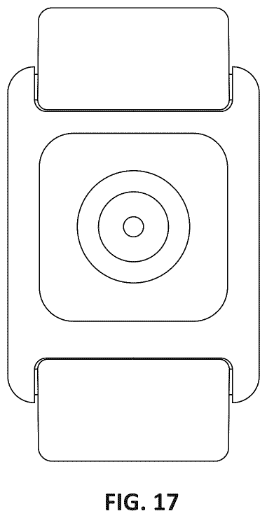

Various embodiments of the wearable device are shown in FIG. 2, FIG. 6, FIG. 10, FIG. 15, FIG. 16 and FIG. 17. As shown in these figures, the wearable device may include a band. In some embodiments, the band may include at least one strap. In some embodiments, the band may include two straps that may be parallel to each other. In some embodiments, the band may include a casing. In some embodiments, the band or casing may be textured. In some embodiments, the user may insert a portion of the biological compartment through an opening formed by the band when the band is in a closed position. In some embodiments, the band may include a first portion and a second portion. In some embodiments, the first portion may include a receiver that may receive the second portion of the band.

In some embodiments, the band may be connected to a tension mechanism. In some embodiments, the tension mechanism may receive the band. The tension mechanism may include at a first slot that can receive a first portion of the band and a second slot that can receive a second portion of the band. In some embodiments, the tension mechanism may implement a winding function to adjust the band. For example, the tension mechanism may be rotated to wrap the band around the tension mechanism, thereby tightening the band or decreasing the length of the band. Similarly, the tension mechanism may be rotated in an opposite direction to unwrap the band from the tension mechanism, thereby loosening the band or increasing the length of the band. In some embodiments, the tension mechanism may be controlled automatically or manually. In some embodiments, the tension mechanism may be round. In some embodiments, the tension mechanism may be knurled to allow the user to better grip the tension mechanism. In some embodiments, the tension mechanism may include a spring. In some embodiments, the spring may be embedded within the band to allow the band to expand. For example, the user may pull the band, causing the spring to lengthen, which thereby may adjust the length of the band. The band may be at its smallest length or circumference when the spring is at its shortest length.

In some embodiments, a portion of the tension mechanism may be positioned within a clasp that may be directly connected to the band. The clasp may allow the band to remain in a closed position when the user is wearing the wearable device. In some embodiments, the clasp may include at least one pulley. The pulley may include a channel that receives a middle portion of the band. For example, a first pulley may include a first channel that may receive a first middle portion of a first portion of the band. Similarly, a second pulley may include a second channel that may receive a second middle portion of a second portion of the band. The first and second middle portion of the band may be positioned within the first and second channel of the pulleys, respectively. A second portion of the tension mechanism may be positioned in parallel to the first portion of the tension mechanism. The parallel configuration may form an opening for the user to insert the biological compartment into. The second portion of the tension mechanism may include a first connection point and a second connection point. The first connection point may be positioned on a first side of the tension mechanism and the second connection point may be positioned on a second side of the tension mechanism. The connection points may serve to connect the band to the tension mechanism. The tension mechanism may further include anchor points to stabilize the band within the tension mechanism. For example, a first end of the first portion of the band may connect to the first connection point and a second end of the first potion of the band may connect to a first anchor point located on the opposite side of the tension mechanism of the first connection point. Similarly, a first end of the second portion of the band may connect to the second connection point and a second end of the second portion of the band may connect to a second anchor point located on the opposite side of the tension mechanism of the second connection point. The tension mechanism may further include knob that may be rotated to tighten or loosen the band. This may cause the band to increase or decrease in circumference.

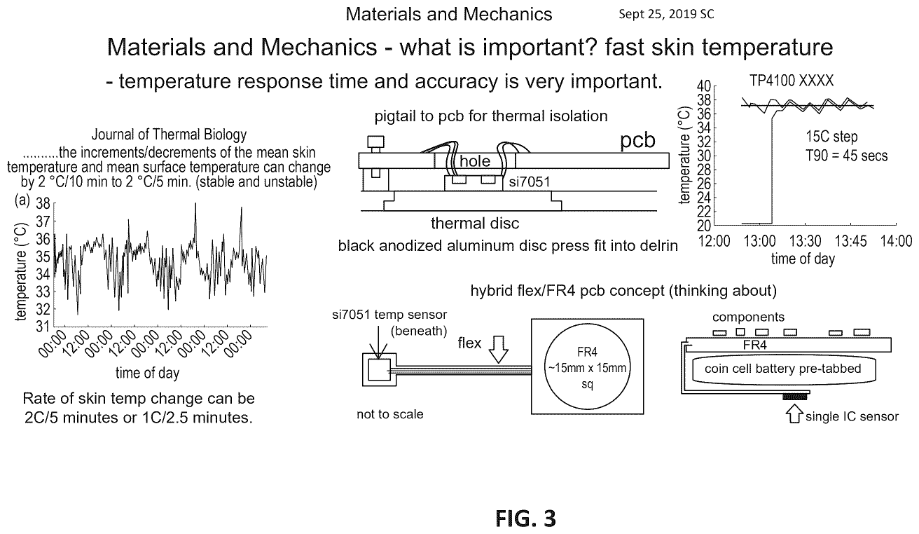

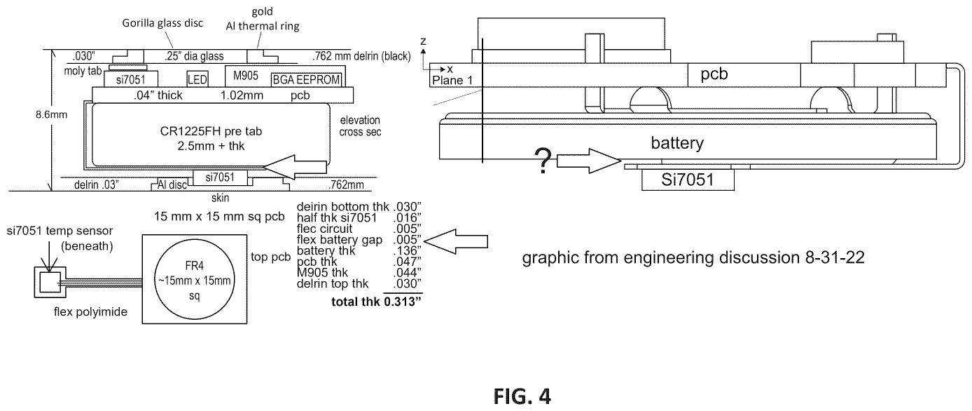

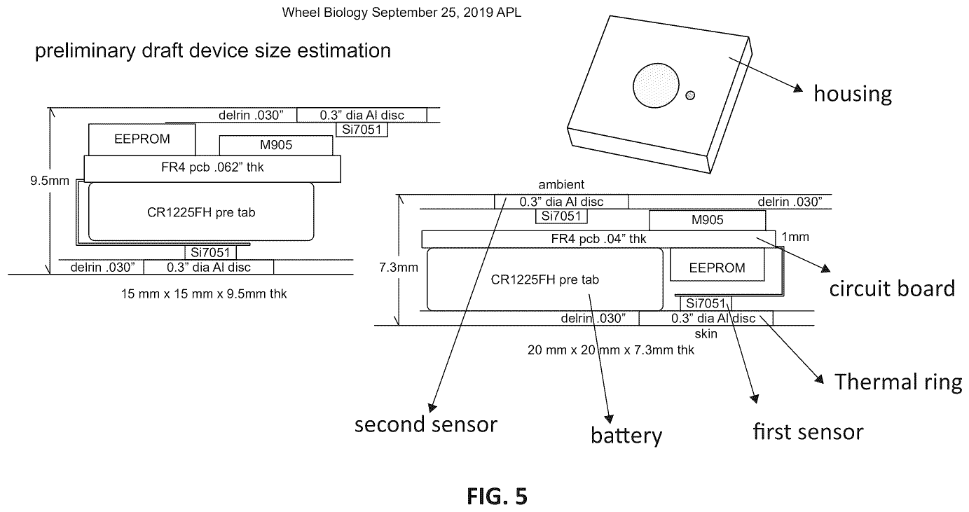

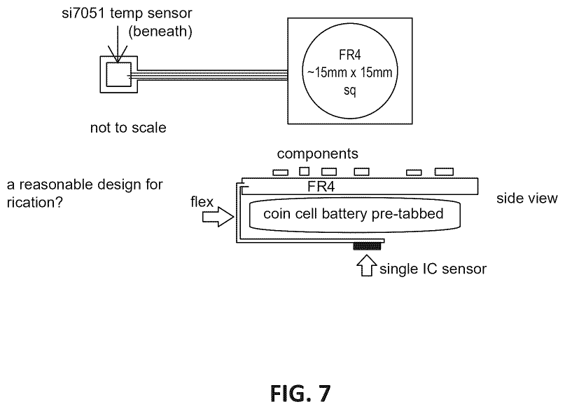

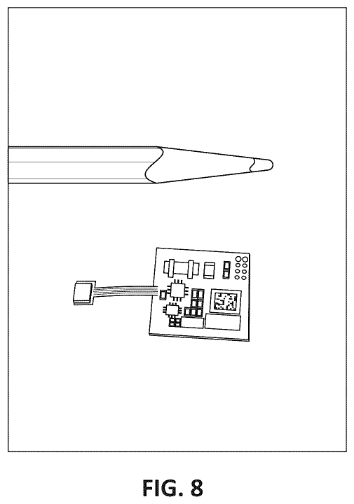

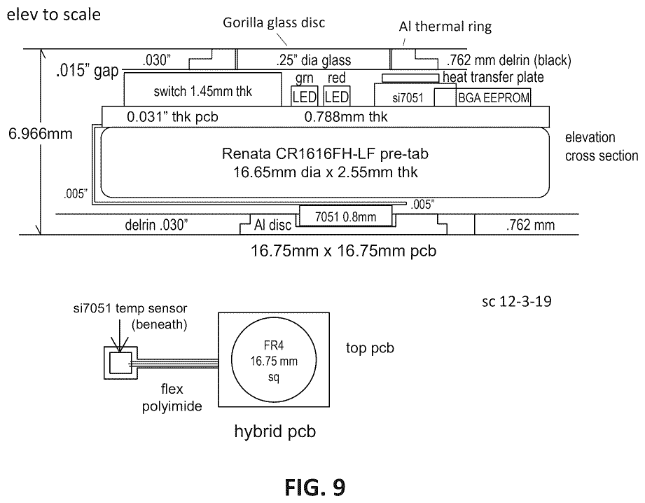

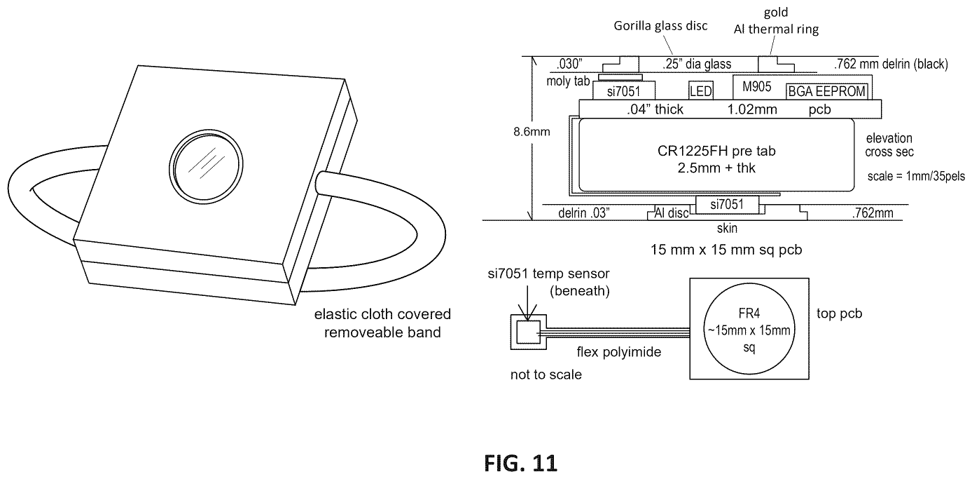

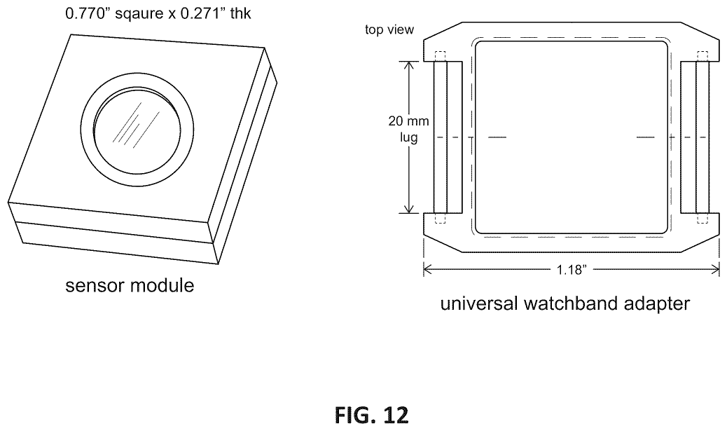

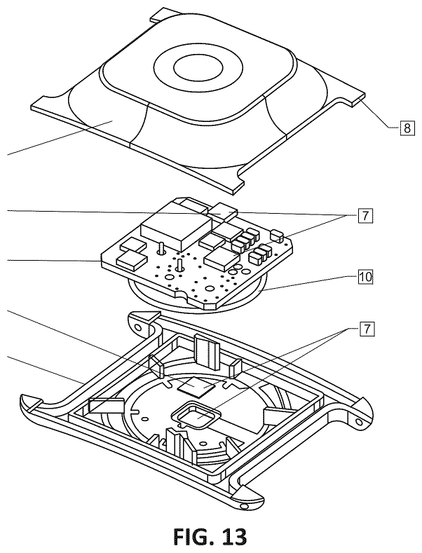

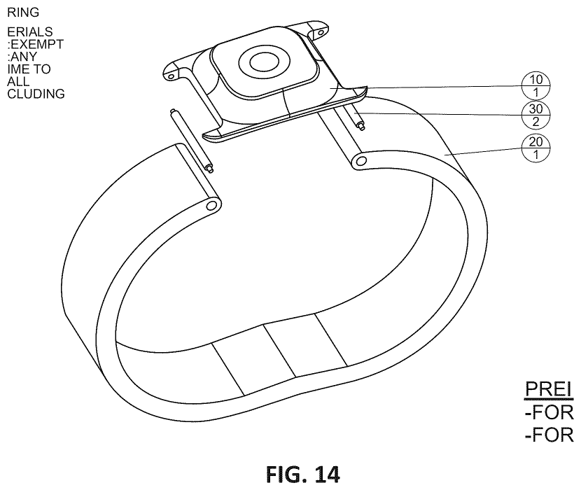

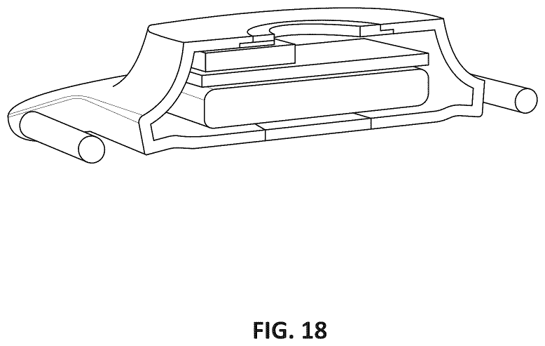

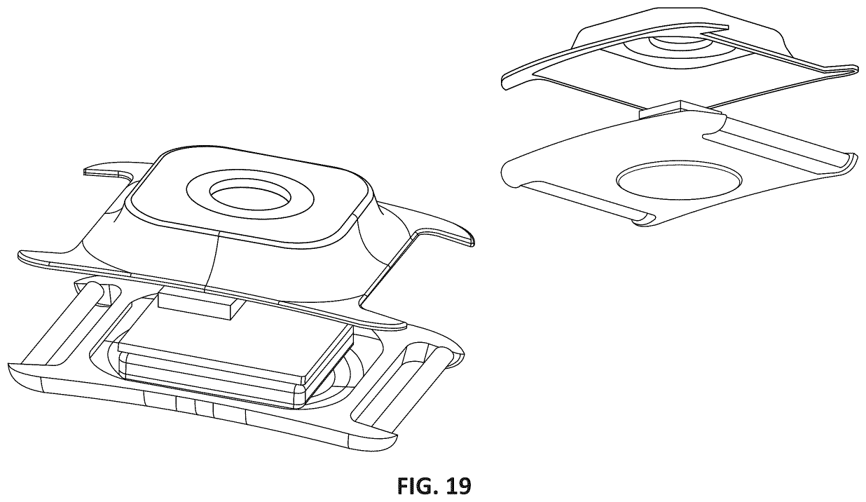

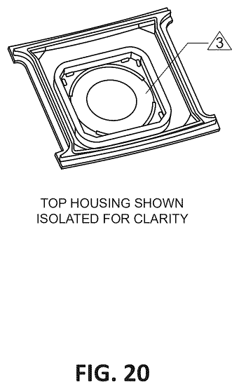

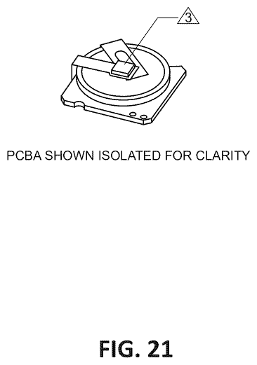

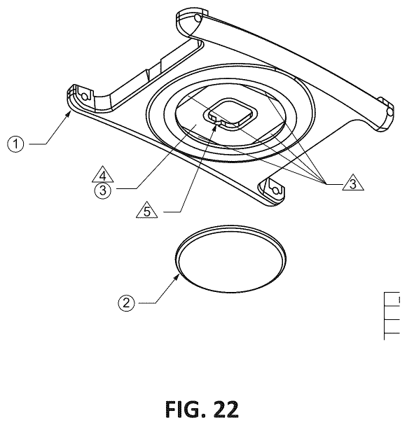

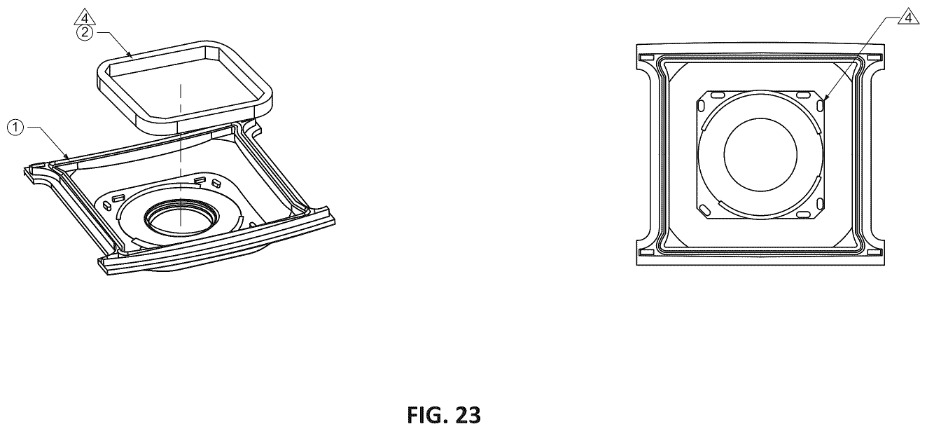

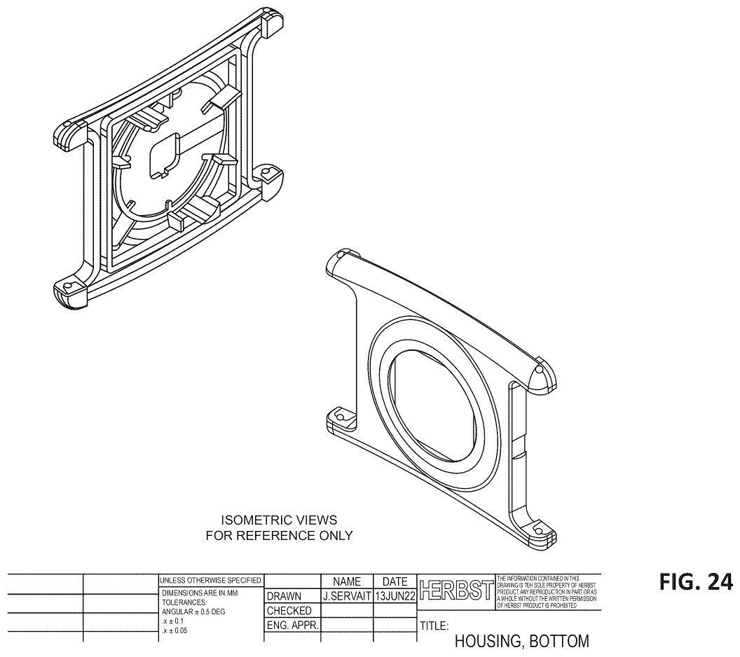

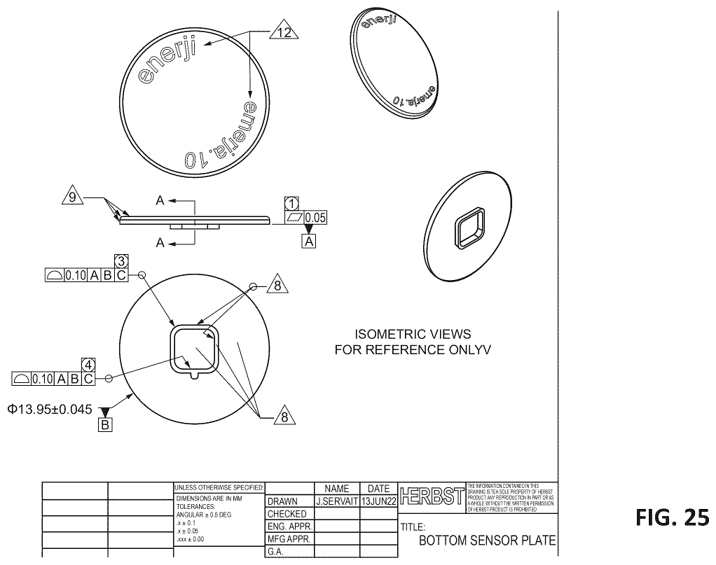

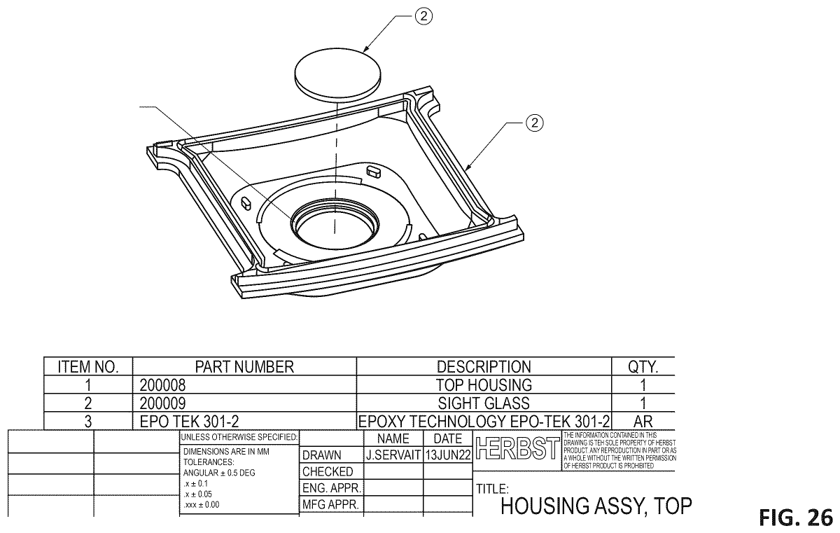

Electronics and other components of the wearable device, and how they are assembled together, are illustrated in FIG. 2, FIG. 3, FIG. 4, FIG. 5, FIG. 7, FIG. 8, FIG. 9, FIG. 11, FIG. 12, FIG. 13, FIG. 14, FIG. 18, FIG. 19, FIG. 20, FIG. 21, FIG. 22, FIG. 23, FIG. 24, FIG. 25 and FIG. 26 in both plan views and cross-sectional views. To obtain the emergent factors of the user, the wearable device may include an array of sensors for measuring user data. In some embodiments, the sensors may record health metrics. In some embodiments, the wearable device continuously records select energy signature metrics or indicators of health for the user. In some embodiments, the wearable device captures emergent-derived complexity at the scale of cell physiology. In some embodiments, the wearable device requires low cost and low power, enabling accessibility and continuous data capture in real-time. In some embodiments, the wearable device comprises a multi-modality sensor system that measures electrochemical, mechanical, structural, thermal, and/or energetic properties reflective of homeostasis and cell physiology. In some embodiments, the wearable device includes sensors that control and/or measure the tension within the tension mechanism. In some embodiments, the sensor may be connected to the band and/or the tension mechanism. In some embodiments, a first sensor may be based on circumferential measurement in which the sensor may dynamically measure the tension within the band and/or the tension within the tension mechanism. In some embodiments, a second sensor may dynamically measure various emergent factors of the biological compartment. For example, the second sensor may dynamically measure the pressure, heat flux, volume of the biological compartment, etc. In some embodiments, the sensor may measure the various emergent factors noninvasively or minimally invasively. In some embodiments, the sensor may dynamically measure the tension in the band and dynamically equate the tension to an internal pressure of the biological compartment. In some embodiments, the sensor may include an embedded microneedle to measure interstitial pressure minimally invasively dynamically. In some embodiments, the sensor may include a controller that may dynamically alter the tension in the tension mechanism and/or band based on the measurements measured by the sensor.

For example, as illustrated in FIG. 5, the wearable device includes a circuit board located within a housing, the circuit board having a first side of the circuit board opposite a second side of the circuit board. The wearable device also includes a first sensor located on the first side of the circuit board and a second sensor located on the second side of the circuit board. The first sensor and the second sensor are thermally decoupled, and the first and second sensors measure said at least one emergent factor of a user. In some embodiments, the first sensor and the second sensor both utilizes a heat sensor (e.g., Si7051 Digital Temperature Sensor from Silicon Labs). The first sensor measures skin temperature, while the second sensor measures ambient temperature. The wearable device further includes a battery that is positioned between the first sensor and the second sensor. Thus, the battery serves to thermally isolate the first sensor from the second sensor. In addition, the second sensor is connected to a thermal ring or a thermal disc that contacts the user's skin when the user is wearing the wearable device. In some embodiments, the device further includes a processor and a firmware.

The wearable device illustrated in FIG. 5 may be used to measure a plurality of data indicative of at least one emergent factor of a biological system, e.g., a user. For example, ambient temperature of the user is measured through a first sensor, and skin temperature of the user is measured through a second sensor, while the first sensor and the second sensor are decoupled. Heat elimination of the user over time can be estimated based on differential ambient temperature, and heat production of the user over time can be estimated based on the measured skin temperature. Thus, basal metabolic status of the user can be estimated based on temporal alignment of heat elimination and heat production.

In addition, a quasiperiodic rhythm of the user can be obtained based on the measured ambient temperature and skin temperature, where the quasiperiodic rhythm is of seconds-timescale, of minutes-timescale, ultradian, circadian, circalunar, or of yearly timescale. A variability of the quasiperiodic rhythm across a predetermined amount of time may be obtained, and thus a health capacity of the user may be determined based on the variability of the quasiperiodic rhythm.

In some embodiments, heat elimination of the user over time can be estimated based on differential ambient temperature, heat production of the user over time can be estimated based on skin temperature, a basal metabolic status of the user can be estimated based on temporal alignment of heat elimination and heat production, and thus a health capacity can be determined by applying a time-dependent function to the estimated basal metabolic status (for example, the time-dependent function is derived from the quasiperiodic rhythm of the user).

In some embodiments, the data relates to the health capacity of the user. In some embodiments, the plurality of data comprises heat flux data. In some embodiments, the data relates to the heat flux of the user. In some embodiments, the data relates to the heat flux of the user over multiple circadian cycles. In some embodiments, at least one health capacity is a basal metabolic status, and at least one emergent factor is a temporal alignment of heat production and heat elimination. In some embodiments, the temporal alignment is related to at least one quasiperiodic rhythm of the user. In some embodiments, the at least one quasiperiodic rhythm is a circadian rhythm.

In some embodiments, the device continuously and contextually characterizes a user's metabolic state by measuring the user's thermal signature and assessing the user's thermoregulatory status. Changes relative to the user's thermoregulatory status are sensitive indicators of change in the user's health state. The device may be configured such that it determines general associations between a user's thermal signature and physiologic reserve. The device may indicate a vital sign of homeostasis or a health signature of the user, and may be utilized in early detection of disease states and management of individual wellness.

### Additional Embodiments

In some applications, the internal temperature sensors are designed and configured to measure temperature outside of device. This is accomplished by, for example, a thermal window. In some embodiments, thermal windows are positioned on both sides of the device—one on the skin side and one on the ambient side. In some embodiments, a thermal window is made of material through which heat can move in both directions (for example, from skin to device, from device to skin; from device to ambient, and from ambient to device) quickly and without distortion. In some embodiments, the sensors are decoupled so that do not contaminate each other. In some embodiments, the windows are decoupled so that do not contaminate each other. In some embodiments, the perimeter of windows cannot be coupled or touched.

While many metals are known to be classic thermal conductors and thus may acts as the thermal window (e.g., a sensor may be attached to a metal window by epoxy), metals are also generally electrically conductive and locks radio signals, limiting the range of Bluetooth signal used in the device. In preferred embodiments, the thermal window also acts as a long-range Bluetooth window. In some embodiments, the thermal window may be made of sapphire or diamond; these materials are dielectric materials but are not electrically conductive, and they have high thermal conductivity.

In preferred embodiments, the material for the thermal window also has low heat absorption. Therefore, preferred materials for the thermal window would also have high thermal diffusivity (the ration between the thermal conductivity and the specific heat by mass, which relates to heat absorption). Thus, diamond and sapphire, being good thermal conductors and having low specific heat (i.e., having a higher thermal diffusivity) are good candidates for the thermal window.

While sapphire is commonly used in optics, optical grade sapphire is expensive. Therefore, in preferred embodiments, the thermal window may be made of synthetic sapphire, e.g., unpolished synthetic sapphire, that is less expensive.

In some embodiments, the thermal window may be a thin sapphire disk. Since sapphire is harder than metal, it can be machined to be thinner than metal. Thinner makes the thermal window better by improving sensor response. Sapphire is also highly biocompatible and would not trigger allergy when touching the skin. In contrast, different kinds of metal may trigger different kinds of allergies. Sapphire can also be made with a wide range of colors. Similarly, hard dielectric materials (whose lattice structure spreads the heat efficiently) can be good candidates for the thermal window, for example, diamond (which is the hardest of known materials) or other gem stones.

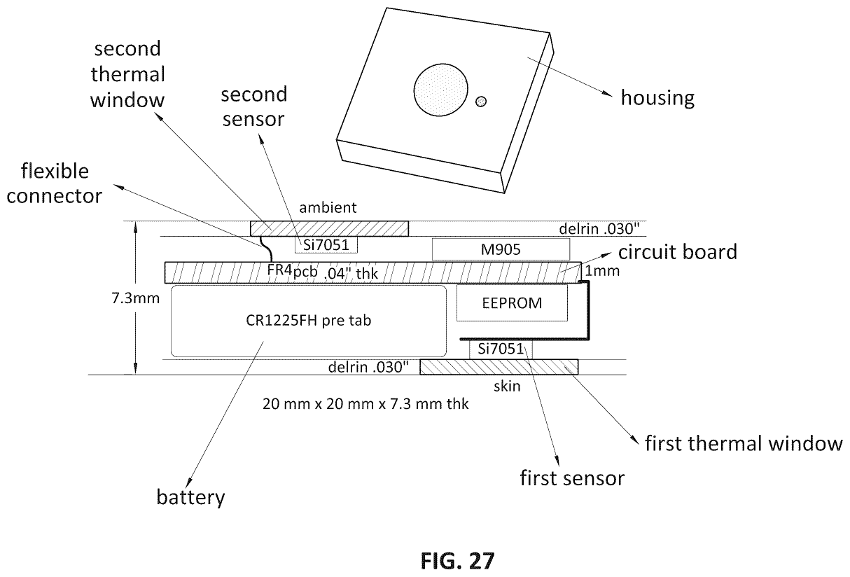

FIG. 27 shows an example wearable device having thermal windows with desired properties as discussed above. The device includes a circuit board located within a housing, the circuit board having a first side of the circuit board opposite a second side of the circuit board. The wearable device also includes a first sensor located on the first side of the circuit board and coupled to a first thermal window, and a second sensor located on the second side of the circuit board and coupled to a second thermal window. The first thermal window and the second thermal window are decoupled. The first sensor and the second sensor are thermally decoupled. The wearable device further includes a battery that is positioned between the first sensor and the second sensor. Thus, the battery serves to thermally isolate the first sensor from the second sensor. In some embodiments, the device further includes a processor and a firmware. In some embodiments, the second sensor is coupled to the circuit board by a flexible connector.

The wearable device illustrated in FIG. 27 may be used to measure a plurality of data indicative of at least one emergent factor of a biological system, e.g., a user. In some embodiments, the first sensor and the second sensor both utilizes a heat sensor (e.g., Si7051 Digital Temperature Sensor from Silicon Labs). In preferred embodiments, the first sensor measures skin temperature, while the second sensor measures ambient temperature. Heat elimination of the user over time can be estimated based on differential ambient temperature, and heat production of the user over time can be estimated based on the measured skin temperature. Thus, basal metabolic status of the user can be estimated based on temporal alignment of heat elimination and heat production.

In some embodiments, in order to improve the thermal response time of the disclosed device, reflective foil, or thermally super-insulating materials made of reflective materials, may be included inside the device to reflect away radiative heat transfer between the sensor plate(s) and the battery/PCB circuit board. Some examples of thermally super-insulating materials made of reflective materials are described in National Aeronautics and Space Administration (NASA) Publication No. NASA CR-2507, “Applications of Aerospace Technology: Reflective Superinsulaton Materials” (January 1975) (available at https://ntrs.nasa.gov/citations/19750006837), the disclosure of which is incorporated herein in its entirety. In some embodiments, in order to improve the thermal response time of the disclosed device, the device enclosure may be designed to have inner and outer walls separated by a mesh of air pockets to minimize thermal transfer through the walls of the device. In some examples, such a design may be achieved by 3D printed structures, such as those described in “Grabowska, B. and Kasperski, J., 2020. The Thermal Conductivity of 3D Printed Plastic Insulation Materials—The Effect of Optimizing the Regular Structure of Closures, Materials, 13(19), p. 4400”, the disclosure of which is incorporated herein in its entirety. Additionally or alternatively, such mesh of air pockets/3D printed structures may be used to minimize heat transfer between the sensor plate(s) and the battery/PCB circuit board.

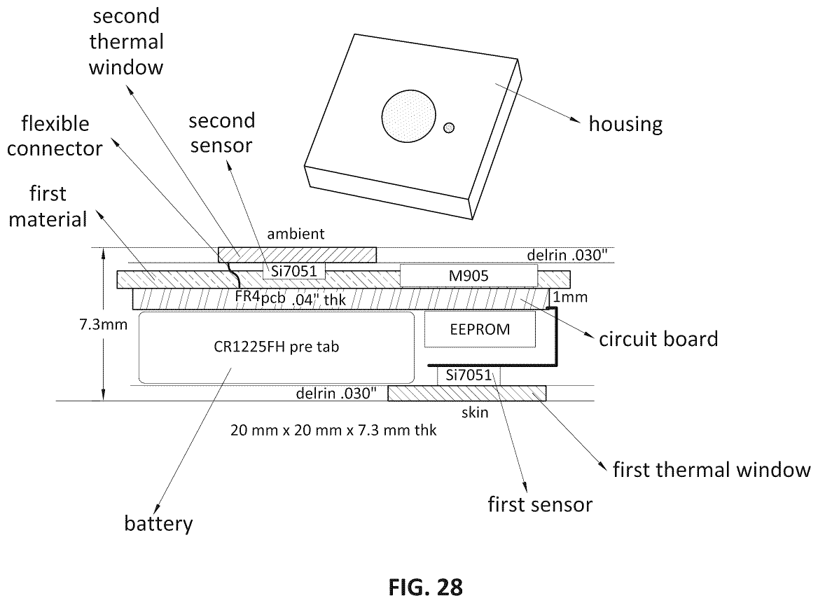

FIG. 28 depicts an example wearable device having an isolation material designed to thermally decouple at least one of the sensors from the circuit board and the battery, as discussed above. In some embodiments, the wearable device includes a housing and a circuit board located within the housing, the circuit board having a first side of the circuit board opposite a second side of the circuit board. In some embodiments, the wearable device includes a first sensor located on the first side of the circuit board, a second sensor located on the second side of the circuit board, and a battery positioned between the first sensor and the second sensor. In some embodiments, the first sensor and the second sensor both utilizes a heat sensor (for example, Si7051 Digital Temperature Sensor from Silicon Labs). In some embodiments, the isolation material has the ability to, at least to a substantial extent, thermally decouple the first sensor from the circuit board and the battery. In some embodiments, the isolation material substantially thermally decouples the second sensor from the circuit board and the battery. In some embodiments, the device further includes a processor and a firmware. In some embodiments, the isolation material substantially thermally decouples the first sensor and the second sensor from the circuit board and the battery. In some embodiments, the isolation material is located on the first side of the circuit board. In some embodiments, the isolation material is located on the second side of the circuit board. In some embodiments, the isolation material is located on the first side of the circuit board and the second side of the circuit board. In some embodiments, the isolation material includes a 3D printed plastic insulation material, a reflective foil or a reflective thermally super-insulating material as described above. In some embodiments, the 3D printed plastic insulation material is configured to minimize thermal transfer between the first sensor and/or the second sensor and the circuit board and the battery. In some embodiments, the reflective foil or the reflective thermally super-insulating material is configured to reflect away radiative heat transfer between the first sensor and/or the second sensor and the circuit board and the battery.

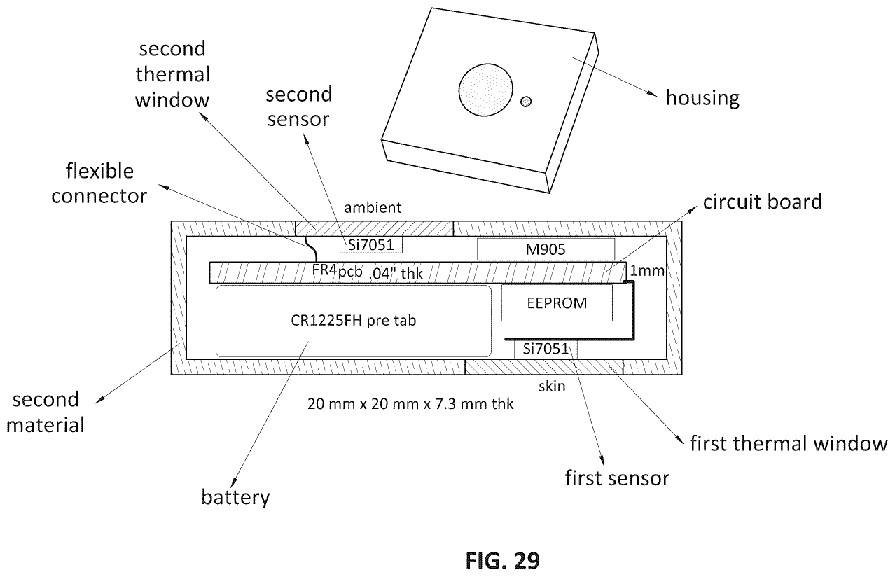

FIG. 29 depicts an example wearable device having an isolation material designed to include a mesh of air pockets that serve to minimize thermal transfer through the walls of the device, as described above. In some embodiments, the wearable device includes a housing and a circuit board located within the housing. In some embodiments, the wearable device further includes a first channel of the circuit board configured to measure skin temperature of the user, and a second channel of the circuit board configured to measure ambient temperature, where the first channel and the second channel are decoupled. In some embodiments, the device further includes a battery, a processor, and a firmware. In some embodiments, the isolation material that includes a mesh of air pockets is configured to minimize thermal transfer through peripheries of the device. In some embodiments, the isolation material is a 3D printed plastic insulation material as described above.

The wearable devices illustrated in FIG. 28 and FIG. 29 may be used to measure a plurality of data indicative of at least one emergent factor of a biological system, e.g., a user. Heat elimination of the user over time can be estimated based on differential ambient temperature, and heat production of the user over time can be estimated based on the measured skin temperature. Thus, basal metabolic status of the user can be estimated based on temporal alignment of heat elimination and heat production.

The embodiments described are examples. Various changes could be made in the above devices and methods without departing from the scope of the invention. All subject matter described in this disclosure, including the accompanying figures, is illustrative and not limiting.

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part incorporated herein by reference in their entireties for the purposes indicated by the context of their citation herein. However, the citation of any document is not to be construed as an admission that it is prior art with respect to the present disclosure.

### FURTHER EMBODIMENTS OF THE DISCLOSED TECHNOLOGY

Embodiment 1. A device configured to measure a plurality of data indicative of at least one emergent factor of a user, the device comprising: a housing; a circuit board located within the housing, the circuit board having a first side of the circuit board opposite a second side of the circuit board; a first sensor located on the first side of the circuit board; and a second sensor located on the second side of the circuit board; wherein the first sensor and the second sensor are decoupled, and wherein the second sensor is coupled to the circuit board by a flexible connector.

Embodiment 2. The device of Embodiment 1, wherein the first sensor is a heat sensor and the second sensor is a heat sensor.

Embodiment 3. The device of Embodiment 1, wherein the first sensor measures skin temperature.

Embodiment 4. The device of Embodiment 1, wherein the second sensor measures ambient temperature.

Embodiment 5. The device of Embodiment 1, further including a battery.

Embodiment 6. The device of Embodiment 1, wherein the first sensor and the second sensor are thermally decoupled.

Embodiment 7. The device of Embodiment 1, wherein the first sensor and the second sensor are configured to measure data indicative of the at least one emergent factor of the user.

Embodiment 8. The device of Embodiment 5, wherein the battery is positioned between the first sensor and the second sensor.

Embodiment 9. The device of Embodiment 5, wherein the battery serves to thermally isolate the first sensor from the second sensor.

Embodiment 10. The device of Embodiment 1, wherein the second sensor is connected to a thermal ring.

Embodiment 11. The device of Embodiment 10, wherein the thermal ring contacts the user.

Embodiment 12. The device of Embodiment 1, further comprising a processor and a firmware.

Embodiment 13. A device configured to measure a plurality of data indicative of at least one emergent factor of a user, the device comprising: a circuit board; a first channel of the circuit board configured to measure ambient temperature; and a second channel of the circuit board configured to measure skin temperature of the user; wherein the first channel and the second channel are decoupled.

Embodiment 14. The device of Embodiment 13, further comprising a battery, a processor, and a firmware.

Embodiment 15. A method of measuring a plurality of data indicative of at least one emergent factor of a user, the method comprising: measuring ambient temperature of the user through a first sensor; and measuring skin temperature of the user through a second sensor, wherein the first sensor and the second sensor are decoupled.

Embodiment 16. The method of Embodiment 15, further comprising: estimating heat elimination of a biological system over time based on differential ambient temperature; estimating heat production of the biological system over time based on skin temperature; and estimating basal metabolic status of the biological system based on temporal alignment of heat elimination and heat production.

Embodiment 17. The method of Embodiment 15, further comprising: obtaining a quasiperiodic rhythm of a biological system based on ambient temperature and skin temperature, wherein the quasiperiodic rhythm is of seconds-timescale, of minutes-timescale, ultradian, circadian, circalunar, or of yearly timescale.

Embodiment 18. The method of Embodiment 17, further comprising: obtaining a variability of the quasiperiodic rhythm across a predetermined amount of time; and determining a health capacity based on the variability of the quasiperiodic rhythm.

Embodiment 19. The method of Embodiment 17, further comprising: estimating heat elimination of a biological system over time based on differential ambient temperature; estimating heat production of the biological system over time based on skin temperature; estimating a basal metabolic status of the biological system based on temporal alignment of heat elimination and heat production; and determining a health capacity by applying a time-dependent function to the estimated basal metabolic status, wherein the time-dependent function is derived from the quasiperiodic rhythm of the biological system.

Embodiment 20. The method of Embodiment 15, wherein the plurality of data comprises heat flux data.

Embodiment 21. The method of Embodiment 20, wherein: at least one health capacity is a basal metabolic status, and at least one emergent factor is a temporal alignment of heat production and heat elimination.

Embodiment 22. The method of Embodiment 21, wherein the temporal alignment is related to at least one quasiperiodic rhythm of a biological system.

Embodiment 23. The method of Embodiment 22, wherein the at least one quasiperiodic rhythm is a circadian rhythm.

Embodiment 24. A device configured to measure a plurality of data indicative of at least one emergent factor of a user, the device comprising: a housing; a circuit board located within the housing, the circuit board having a first side of the circuit board opposite a second side of the circuit board; a first sensor located on the first side of the circuit board and coupled to a first thermal window; and a second sensor located on the second side of the circuit board and coupled to a second thermal window; wherein the first sensor and the second sensor are decoupled, wherein the first thermal window and the second thermal window are decoupled, and wherein the second sensor is coupled to the circuit board by a flexible connector.

Embodiment 25. The device of Embodiment 24, wherein the first sensor is a heat sensor and the second sensor is a heat sensor.

Embodiment 26. The device of Embodiment 24, wherein the first sensor measures skin temperature.

Embodiment 27. The device of Embodiment 24, wherein the second sensor measures ambient temperature.

Embodiment 28. The device of Embodiment 24, wherein the first thermal window contacts the user and the second thermal window contacts ambient air.

Embodiment 29. The device of Embodiment 28, wherein the first thermal window and the second thermal window are thermally conductive and permit Bluetooth signals to be transmitted through the device.

Embodiment 30. The device of Embodiment 29, wherein the first thermal window and the second thermal window have a thickness configured to permit an efficient first sensor response and an efficient second sensor response.

Embodiment 31. The device of Embodiment 29, wherein the first thermal window and the second thermal window are made of a dielectric material.

Embodiment 32. The device of Embodiment 31, wherein the dielectric material is sapphire.

Embodiment 33. The device of Embodiment 31, wherein the dielectric material is diamond.

Embodiment 34. The device of one or more of Embodiments 32 and 33, wherein the dielectric material is synthetic.

Embodiment 35. The device of Embodiment 24, further including a battery.

Embodiment 36. The device of Embodiment 24, wherein the first sensor and the second sensor are thermally decoupled.

Embodiment 37. The device of Embodiment 24, wherein the first sensor and the second sensor are configured to measure data indicative of the at least one emergent factor of the user.

Embodiment 38. The device of Embodiment 35, wherein the battery is positioned between the first sensor and the second sensor.

Embodiment 39. The device of Embodiment 35, wherein the battery serves to thermally isolate the first sensor from the second sensor.

Embodiment 40. The device of Embodiment 24, further comprising a processor and a firmware.

Embodiment 41. A device configured to measure a plurality of data indicative of at least one emergent factor of a user, the device comprising: a circuit board; a first channel of the circuit board configured to measure ambient temperature; and a second channel of the circuit board configured to measure skin temperature of the user; wherein the first channel and the second channel are decoupled, and wherein the first channel is coupled to a first thermal window that contacts the user and the second channel is coupled to a second thermal window that contacts ambient air.

Embodiment 42. The device of Embodiment 41, further comprising a battery, a processor, and a firmware.

Embodiment 43. The device of Embodiment 41, wherein the first thermal window and the second thermal window are decoupled.

Embodiment 44. The device of Embodiment 43, wherein the first thermal window and the second thermal window are made of a dielectric material.

Embodiment 45. A method of measuring a plurality of data indicative of at least one emergent factor of a user, the method comprising: measuring ambient temperature of the user through a first sensor; and measuring skin temperature of the user through a second sensor, wherein the first sensor and the second sensor are decoupled, and wherein the first sensor is coupled to a first thermal window and the second sensor is coupled to a second thermal window.

Embodiment 46. The method of Embodiment 45, further comprising: estimating heat elimination of a biological system over time based on differential ambient temperature; estimating heat production of the biological system over time based on skin temperature; and estimating basal metabolic status of the biological system based on temporal alignment of heat elimination and heat production.

Embodiment 47. The method of Embodiment 45, further comprising: obtaining a quasiperiodic rhythm of a biological system based on ambient temperature and skin temperature, wherein the quasiperiodic rhythm is of seconds-timescale, of minutes-timescale, ultradian, circadian, circalunar, or of yearly timescale.

Embodiment 48. The method of Embodiment 47, further comprising: obtaining a variability of the quasiperiodic rhythm across a predetermined amount of time; and determining a health capacity based on the variability of the quasiperiodic rhythm.

Embodiment 49. The method of Embodiment 47, further comprising: estimating heat elimination of a biological system over time based on differential ambient temperature; estimating heat production of the biological system over time based on skin temperature; estimating a basal metabolic status of the biological system based on temporal alignment of heat elimination and heat production; and determining a health capacity by applying a time-dependent function to the estimated basal metabolic status, wherein the time-dependent function is derived from the quasiperiodic rhythm of the biological system.

Embodiment 50. The method of Embodiment 45, wherein the plurality of data comprises heat flux data.

Embodiment 51. The method of Embodiment 50, wherein: at least one health capacity is a basal metabolic status, and at least one emergent factor is a temporal alignment of heat production and heat elimination.

Embodiment 52. The method of Embodiment 51, wherein the temporal alignment is related to at least one quasiperiodic rhythm of a biological system.

Embodiment 53. The method of Embodiment 52, wherein the at least one quasiperiodic rhythm is a circadian rhythm.

Embodiment 54. A device configured to measure a plurality of data indicative of at least one emergent factor of a user, the device comprising: a housing; a circuit board located within the housing, the circuit board having a first side of the circuit board opposite a second side of the circuit board; a first sensor located on the first side of the circuit board; a second sensor located on the second side of the circuit board; and a battery positioned between the first sensor and the second sensor, wherein a first isolation material thermally decouples the first sensor and/or the second sensor from the circuit board and the battery.

Embodiment 55. The device of Embodiment 54, wherein the first sensor is coupled to a first thermal window.

Embodiment 56. The device of Embodiment 54, wherein the second sensor is coupled to a second thermal window.

Embodiment 57. The device of Embodiment 56, wherein the first thermal window and the second thermal window are decoupled.

Embodiment 58. The device of Embodiment 56, wherein the first thermal window contacts the user and the second thermal window contacts ambient air.

Embodiment 59. The device of Embodiment 56, wherein the first thermal window and the second thermal window are thermally conductive and permit Bluetooth signals to be transmitted through the device.

Embodiment 60. The device of Embodiment 56, wherein the first thermal window and the second thermal window have a thickness configured to permit an efficient first sensor response and an efficient second sensor response.

Embodiment 61. The device of Embodiment 56, wherein the first thermal window and the second thermal window are made of a dielectric material.

Embodiment 62. The device of Embodiment 61, wherein the dielectric material is sapphire.

Embodiment 63. The device of Embodiment 61, wherein the dielectric material is diamond.

Embodiment 64. The device of one or more of Embodiments 62 and 63, wherein the dielectric material is synthetic.

Embodiment 65. The device of Embodiment 54, wherein the first sensor is a heat sensor and the second sensor is a heat sensor.

Embodiment 66. The device of Embodiment 54, wherein the first sensor and the second sensor are thermally decoupled.

Embodiment 67. The device of Embodiment 54, wherein the first sensor measures skin temperature.

Embodiment 68. The device of Embodiment 54, wherein the second sensor measures ambient temperature.

Embodiment 69. The device of Embodiment 54, wherein the first sensor and the second sensor are configured to measure data indicative of the at least one emergent factor of the user.

Embodiment 70. The device of Embodiment 54, further comprising a processor and a firmware.

Embodiment 71. The device of Embodiment 54, wherein the first isolation material is located on the first side of the circuit board and/or on the second side of the circuit board.

Embodiment 72. The device of Embodiment 54, wherein the first isolation material comprises: a 3D printed plastic insulation material, a reflective foil, a reflective thermally super-insulating material, or any combination thereof.

Embodiment 73. The device of Embodiment 72, wherein the 3D printed plastic insulation material is configured to minimize thermal transfer between the first sensor and/or the second sensor and the circuit board and the battery.

Embodiment 74. The device of Embodiment 72, wherein the reflective foil and/or the reflective thermally super-insulating material is configured to reflect away radiative heat transfer between the first sensor and/or the second sensor and the circuit board and the battery.

Embodiment 75. The device of Embodiment 54, wherein at least a face of the housing comprises: a second isolation material, a mesh of air pockets, or any combination thereof.

Embodiment 76. The device of Embodiment 75, wherein the second isolation material and/or the mesh of air pockets is configured to minimize thermal transfer through peripheries of the device.

Embodiment 77. The device of Embodiment 75, wherein the second isolation material is a 3D printed plastic insulation material.

Embodiment 78. A device configured to measure a plurality of data indicative of at least one emergent factor of a user, the device comprising: a housing; a circuit board located within the housing; a first channel of the circuit board configured to measure skin temperature of the user; and a second channel of the circuit board configured to measure ambient temperature; wherein the first channel and the second channel are decoupled, and wherein at least a face of the housing comprises: an isolation material, a mesh of air pockets, or any combination thereof.

Embodiment 79. The device of Embodiment 78, wherein the isolation material and/or the mesh of air pockets is configured to minimize thermal transfer through peripheries of the device.

Embodiment 80. The device of Embodiment 78, wherein the isolation material is a 3D printed plastic insulation material.

Embodiment 81. The device of Embodiment 78, further comprising: a battery, a processor, a firmware, or any combination thereof.

Embodiment 82. The device of Embodiment 78, wherein the first channel is coupled to a first thermal window that contacts the user and the second channel is coupled to a second thermal window that contacts ambient air.

Embodiment 83. The device of Embodiment 82, wherein the first thermal window and the second thermal window are decoupled.

Embodiment 84. The device of Embodiment 82, wherein the first thermal window and the second thermal window are thermally conductive and permit Bluetooth signals to be transmitted through the device.

Embodiment 85. The device of Embodiment 82, wherein the first thermal window and the second thermal window have a thickness configured to permit an efficient first sensor response and an efficient second sensor response.

Embodiment 86. The device of Embodiment 82, wherein the first thermal window and the second thermal window are made of a dielectric material.

Embodiment 87. The device of Embodiment 86, wherein the dielectric material is sapphire.

Embodiment 88. The device of Embodiment 86, wherein the dielectric material is diamond.

Embodiment 89. The device of one or more of Embodiments 87 and 88, wherein the dielectric material is synthetic.

Embodiment 90. The device of Embodiment 82, wherein a first sensor is coupled to the first thermal window.

Embodiment 91. The device of Embodiment 90, wherein a second sensor is coupled to the second thermal window.

Embodiment 92. The device of Embodiment 90, wherein the first sensor is a heat sensor and the second sensor is a heat sensor.

Embodiment 93. The device of Embodiment 90, wherein the first sensor and the second sensor are thermally decoupled.

Embodiment 94. The device of Embodiment 90, wherein the first sensor measures skin temperature.

Embodiment 95. The device of Embodiment 90, wherein the second sensor measures ambient temperature.

Embodiment 96. The device of Embodiment 90, wherein the first sensor and the second sensor are configured to measure data indicative of the at least one emergent factor of the user.

Embodiment 97. A method of measuring a plurality of data indicative of at least one emergent factor of a user, the method comprising:
measuring ambient temperature and skin temperature of the user using the device according to any of Embodiments 54-96.

Embodiment 98. The method of Embodiment 97, further comprising: estimating heat elimination of a biological system over time based on differential ambient temperature; estimating heat production of the biological system over time based on skin temperature; and estimating basal metabolic status of the biological system based on temporal alignment of heat elimination and heat production.

Embodiment 99. The method of Embodiment 97, further comprising: obtaining a quasiperiodic rhythm of a biological system based on ambient temperature and skin temperature, wherein the quasiperiodic rhythm is of seconds-timescale, of minutes-timescale, ultradian, circadian, circalunar, or of yearly timescale.

Embodiment 100. The method of Embodiment 99, further comprising: obtaining a variability of the quasiperiodic rhythm across a predetermined amount of time; and determining a health capacity based on the variability of the quasiperiodic rhythm.

Embodiment 101. The method of Embodiment 99, further comprising: estimating heat elimination of a biological system over time based on differential ambient temperature; estimating heat production of the biological system over time based on skin temperature; estimating a basal metabolic status of the biological system based on temporal alignment of heat elimination and heat production; and determining a health capacity by applying a time-dependent function to the estimated basal metabolic status, wherein the time-dependent function is derived from the quasiperiodic rhythm of the biological system.

Embodiment 102. The method of Embodiment 97, wherein the plurality of data comprises heat flux data.

Embodiment 103. The method of Embodiment 102, wherein: at least one health capacity is a basal metabolic status, and at least one emergent factor is a temporal alignment of heat production and heat elimination.

Embodiment 104. The method of Embodiment 103, wherein the temporal alignment is related to at least one quasiperiodic rhythm of a biological system.

Embodiment 105. The method of Embodiment 104, wherein the at least one quasiperiodic rhythm is a circadian rhythm.

What is claimed is:

1. A device configured to measure a plurality of data indicative of at least one emergent factor of a user, the device comprising:
a housing;
a circuit board located within the housing, the circuit board having a first side of the circuit board opposite a second side of the circuit board;
a first sensor located on the first side of the circuit board and coupled to a first thermal window; and
a second sensor located on the second side of the circuit board and coupled to a second thermal window;
wherein the first sensor and the second sensor are decoupled,
wherein the first thermal window and the second thermal window are decoupled, and
wherein the second sensor is coupled to the circuit board by a flexible connector.

2. The device of claim 1, wherein the first sensor is a heat sensor and the second sensor is a heat sensor.

3. The device of claim 1, wherein the first sensor measures skin temperature.

4. The device of claim 1, wherein the second sensor measures ambient temperature.

5. The device of claim 1, wherein the first thermal window contacts the user and the second thermal window contacts ambient air.

6. The device of claim 5, wherein the first thermal window and the second thermal window are thermally conductive and permit Bluetooth signals to be transmitted through the device.

7. The device of claim 6, wherein the first thermal window and the second thermal window are made of a dielectric material.

8. The device of claim 7, wherein the dielectric material is sapphire.

9. The device of claim 7, wherein the dielectric material is diamond.

10. The device of one or more of claims 8 and 9, wherein the dielectric material is synthetic.

11. The device of claim 1, further including a battery.

12. The device of claim 1, wherein the first sensor and the second sensor are thermally decoupled.

13. The device of claim 1, wherein the first sensor and the second sensor are configured to measure data indicative of the at least one emergent factor of the user.

14. The device of claim 11, wherein the battery is positioned between the first sensor and the second sensor.

15. The device of claim 11, wherein the battery serves to thermally isolate the first sensor from the second sensor.

16. The device of claim 1, further comprising a processor and a firmware.

17. A device configured to measure a plurality of data indicative of at least one emergent factor of a user, the device comprising:
a circuit board;
a first channel of the circuit board configured to measure skin temperature of the user; and
a second channel of the circuit board configured to measure ambient temperature;
wherein the first channel and the second channel are decoupled, and
wherein the first channel is coupled to a first thermal window that contacts the user and the second channel is coupled to a second thermal window that contacts ambient air.

18. The device of claim 17, further comprising a battery, a processor, and a firmware.

19. The device of claim 17, wherein the first thermal window and the second thermal window are decoupled.

20. The device of claim 19, wherein the first thermal window and the second thermal window are made of a dielectric material.

21. A method of measuring a plurality of data indicative of at least one emergent factor of a user, the method comprising:
measuring ambient temperature of the user through a first sensor; and
measuring skin temperature of the user through a second sensor,
wherein the first sensor and the second sensor are decoupled, and
wherein the first sensor is coupled to a first thermal window and the second sensor is coupled to a second thermal window.

22. The method of claim 21, further comprising:
estimating heat elimination of a biological system over time based on differential ambient temperature;
estimating heat production of the biological system over time based on skin temperature; and
estimating basal metabolic status of the biological system based on temporal alignment of heat elimination and heat production.

23. The method of claim 21, further comprising:
obtaining a quasiperiodic rhythm of a biological system based on ambient temperature and skin temperature, wherein the quasiperiodic rhythm is of seconds-timescale, of minutes-timescale, ultradian, circadian, circalunar, or of yearly timescale.

24. The method of claim 23, further comprising:
obtaining a variability of the quasiperiodic rhythm across a predetermined amount of time; and
determining a health capacity based on the variability of the quasiperiodic rhythm.

25. The method of claim 23, further comprising:
estimating heat elimination of a biological system over time based on differential ambient temperature;
estimating heat production of the biological system over time based on skin temperature;
estimating a basal metabolic status of the biological system based on temporal alignment of heat elimination and heat production; and
determining a health capacity by applying a time-dependent function to the estimated basal metabolic status, wherein the time-dependent function is derived from the quasiperiodic rhythm of the biological system.

26. The method of claim 21, wherein the plurality of data comprises heat flux data.

27. The method of claim 26, wherein:
at least one health capacity is a basal metabolic status, and
at least one emergent factor is a temporal alignment of heat production and heat elimination.

28. The method of claim 27, wherein the temporal alignment is related to at least one quasiperiodic rhythm of a biological system.

29. The method of claim 28, wherein the at least one quasiperiodic rhythm is a circadian rhythm.

30. A device configured to measure a plurality of data indicative of at least one emergent factor of a user, the device comprising:
a housing;
a circuit board located within the housing, the circuit board having a first side of the circuit board opposite a second side of the circuit board;
a first sensor located on the first side of the circuit board;
a second sensor located on the second side of the circuit board; and
a battery positioned between the first sensor and the second sensor,
wherein a first isolation material thermally decouples the first sensor and/or the second sensor from the circuit board and the battery.

31. The device of claim 30, wherein the first sensor is coupled to a first thermal window.

32. The device of claim 30, wherein the second sensor is coupled to a second thermal window.

33. The device of claim 32, wherein the first thermal window and the second thermal window are decoupled.

34. The device of claim 32, wherein the first thermal window contacts the user and the second thermal window contacts ambient air.

35. The device of claim 32, wherein the first thermal window and the second thermal window are thermally conductive and permit Bluetooth signals to be transmitted through the device.

36. The device of claim 32, wherein the first thermal window and the second thermal window are made of a dielectric material.

37. The device of claim 36, wherein the dielectric material is sapphire.

38. The device of claim 36, wherein the dielectric material is diamond.

39. The device of one or more of claims 37 and 38, wherein the dielectric material is synthetic.

40. The device of claim 30, wherein the first sensor is a heat sensor and the second sensor is a heat sensor.

41. The device of claim 30, wherein the first sensor and the second sensor are thermally decoupled.

42. The device of claim 30, wherein the first sensor measures skin temperature.

43. The device of claim 30, wherein the second sensor measures ambient temperature.

44. The device of claim 30, wherein the first sensor and the second sensor are configured to measure data indicative of the at least one emergent factor of the user.

45. The device of claim 30, further comprising a processor and a firmware.

46. The device of claim 30, wherein the first isolation material is located on the first side of the circuit board and/or on the second side of the circuit board.

47. The device of claim 30, wherein the first isolation material comprises: a 3D printed plastic insulation material, a reflective foil, a reflective thermally super-insulating material, or any combination thereof.

48. The device of claim 47, wherein the 3D printed plastic insulation material is configured to minimize thermal transfer between the first sensor and/or the second sensor and the circuit board and the battery.

49. The device of claim 47, wherein the reflective foil and/or the reflective thermally super-insulating material is configured to reflect away radiative heat transfer between the first sensor and/or the second sensor and the circuit board and the battery.

50. The device of claim 30, wherein at least a face of the housing comprises: a second isolation material, a mesh of air pockets, or any combination thereof.

51. The device of claim 50, wherein the second isolation material and/or the mesh of air pockets is configured to minimize thermal transfer through peripheries of the device.

52. The device of claim 50, wherein the second isolation material is a 3D printed plastic insulation material.

53. A device configured to measure a plurality of data indicative of at least one emergent factor of a user, the device comprising:
a housing;
a circuit board located within the housing;
a first channel of the circuit board configured to measure skin temperature of the user; and
a second channel of the circuit board configured to measure ambient temperature;
wherein the first channel and the second channel are decoupled, and
wherein at least a face of the housing comprises: an isolation material, a mesh of air pockets, or any combination thereof.

54. The device of claim 53, wherein the isolation material and/or the mesh of air pockets is configured to minimize thermal transfer through peripheries of the device.

55. The device of claim 53, wherein the isolation material is a 3D printed plastic insulation material.

56. The device of claim 53, further comprising: a battery, a processor, a firmware, or any combination thereof.

57. The device of claim 53, wherein the first channel is coupled to a first thermal window that contacts the user and the second channel is coupled to a second thermal window that contacts ambient air.

58. The device of claim 57, wherein the first thermal window and the second thermal window are decoupled.

59. The device of claim 57, wherein the first thermal window and the second thermal window are thermally conductive and permit Bluetooth signals to be transmitted through the device.

60. The device of claim 57, wherein the first thermal window and the second thermal window are made of a dielectric material.

61. The device of claim 60, wherein the dielectric material is sapphire.

62. The device of claim 60, wherein the dielectric material is diamond.

63. The device of one or more of claims 61 and 62, wherein the dielectric material is synthetic.

64. The device of claim 57, wherein a first sensor is coupled to the first thermal window.

65. The device of claim 64, wherein a second sensor is coupled to the second thermal window.

66. The device of claim 64, wherein the first sensor is a heat sensor and the second sensor is a heat sensor.

67. The device of claim 64, wherein the first sensor and the second sensor are thermally decoupled.

68. The device of claim 64, wherein the first sensor measures skin temperature.

69. The device of claim 64, wherein the second sensor measures ambient temperature.

70. The device of claim 64, wherein the first sensor and the second sensor are configured to measure data indicative of the at least one emergent factor of the user.

71. A method of measuring a plurality of data indicative of at least one emergent factor of a user, the method comprising:
measuring ambient temperature and skin temperature of the user using the device according to any of claims 30-70.

72. The method of claim 71, further comprising:
estimating heat elimination of a biological system over time based on differential ambient temperature;
estimating heat production of the biological system over time based on skin temperature; and
estimating basal metabolic status of the biological system based on temporal alignment of heat elimination and heat production.

73. The method of claim 71, further comprising:
obtaining a quasiperiodic rhythm of a biological system based on ambient temperature and skin temperature, wherein the quasiperiodic rhythm is of seconds-timescale, of minutes-timescale, ultradian, circadian, circalunar, or of yearly timescale.

74. The method of claim 73, further comprising:
obtaining a variability of the quasiperiodic rhythm across a predetermined amount of time; and
determining a health capacity based on the variability of the quasiperiodic rhythm.

75. The method of claim 73, further comprising:
estimating heat elimination of a biological system over time based on differential ambient temperature;
estimating heat production of the biological system over time based on skin temperature;
estimating a basal metabolic status of the biological system based on temporal alignment of heat elimination and heat production; and
determining a health capacity by applying a time-dependent function to the estimated basal metabolic status, wherein the time-dependent function is derived from the quasiperiodic rhythm of the biological system.

76. The method of claim 73, wherein the plurality of data comprises heat flux data.

77. The method of claim 76, wherein:
at least one health capacity is a basal metabolic status, and
at least one emergent factor is a temporal alignment of heat production and heat elimination.

78. The method of claim 77, wherein the temporal alignment is related to at least one quasiperiodic rhythm of a biological system.

79. The method of claim 78, wherein the at least one quasiperiodic rhythm is a circadian rhythm.
